(19) United States
(12) Patent Application Publication
(10) Pub. No.: US 2023/0293163 A1
(43) Pub. Date: Sep. 21, 2023

(54) MEDICAL RETRACTOR SUITABLE FOR ELECTROCAUTERIZATION PROCEDURES

(71) Applicant: Daniel A. Burns, Warren, PA (US)

(72) Inventor: Daniel A. Burns, Warren, PA (US)

(21) Appl. No.: 18/184,103

(22) Filed: Mar. 15, 2023

Related U.S. Application Data
(60) Provisional application No. 63/269,615, filed on Mar. 19, 2022.

(51) Int. Cl. A61B 17/02

(52) U.S. Cl. CPC A61B 17/02; A61B 2018/00083

(57) ABSTRACT

Medical retractors for providing access to a natural or clinician-formed body cavity are provided. An example of such a medical retractor may include an elongated handle member having a distal end and a proximal end, the elongated handle member configured to be held in a clinician's hand, a retractor blade configured for insertion into a body cavity and blade-handle coupling mechanism that rotatably couples a distal end of the elongated handle member and to a proximal end of the retractor blade. The elongated handle member can be rotated to improve access to the body cavity. Related medical retractor kits and methods are operation are also provided.

### RELATED APPLICATIONS

The present application claims priority to U.S. Provisional Patent Application No. 63/269,615, filed Mar. 19, 2022, entitled “MEDICAL RETRACTOR SUITABLE FOR ELECTROCAUTERIZATION PROCEDURES”, hereby incorporated by reference in its entirety.

### TECHNICAL FIELD

The present disclosure relates to a medical retractor for providing access to a cavity of a human body, such as a vagina, for examination and/or intervention.

### BACKGROUND

Medical procedures in body cavities often presents challenges of access. To gain adequate visualization of relevant structures, medical assistants often have to assume awkward positioning with inadequate stability of handheld retractors. This may make it difficult to maintain a desired position for longer periods of time. Furthermore, because the medical assistant needs to maintain the retractor in place, the hands and arms of the medical assistant may present an obstacle to the to the surgeon or clinician performing a diagnostic or surgical procedure.

Medical retractors may be used to provide access to surgical procedures involving the use of various implements at the surgical site. Such implements may include devices such as electrocauterization devices that rely on electricity to generate heat to remove tissue or to seal tissue. Such electrocauterization procedures can be complicated by the use of typical retractors which may cause complications by conducting heat from the surgical site to other areas that are not intended to be treated and may be damaged by excessive heat distributed by conductive retractor blades.

### SUMMARY

An embodiment of the present disclosure includes a medical retractor that includes an elongated handle member having a distal end and a proximal end, a retractor blade configured for insertion into a body cavity, the retractor blade having a distal end and a proximal end, and a blade-handle coupling mechanism that rotatably couples a distal end of the elongated handle member and to a proximal end of the retractor blade. The elongated handle member may be configured be handheld when the medical retractor is in use.

Some embodiments of the medical retractor may include one or more of the following features. The elongated handle member may have a substantially straight portion at the proximal end and a curved portion at the distal end. The curved portion at the distal end may be a J-curve. An axis of the substantially straight portion and an axis at a distal end of the curved portion may be approximately orthogonal. The blade-handle coupling mechanism may permit rotation of the retractor blade about an axis at a distal end of the curved portion of the elongated handle member.

Some embodiments of the medical retractor may include one or more of the following features. The blade-handle coupling mechanism may include at least one detent that secures orientation of an axis of the elongated handle member with an axis of the retractor blade. The blade-handle coupling mechanism may further include a plurality of detents that secure orientation of the axis of the elongated handle member with the axis of the retractor blade in a plurality of angles. The plurality of angles may include at least approximately 0 degrees and approximately 180 degrees. The plurality of angles may include approximately 0 degrees and another angle substantially less than 180 degrees. The plurality of angles may include at least two of ±60 degrees, ±90 degrees, ±120 degrees, ±135 degrees, ±150 degrees, and 180 degrees.

Some embodiments of the medical retractor may include one or more of the following features. The retractor blade may be formed from a non-conductive material such that the retractor blade includes at least a non-conductive outer surface. The non-conductive outer surface includes or is formed from an electrically non-conductive material. The non-conductive outer surface may include a thermally non-conductive material. The non-conductive outer surface may be formed of a polycarbonate material, a polymethyl methacrylate material, or a ceramic material. The retractor blade may be formed of a non-conductive material and the elongated handle member may be formed from a material is that different than the non-conductive material of the retractor blade. The retractor blade may be curved in a transverse cross-section. The retractor blade is curved along a longitudinal axis. The curve of the retractor blade may be such that the retractor blade does not contact the elongated handle member when the retractor blade and the elongated handle member form an angle about approximately 0 degrees.

Some embodiments of the medical retractor may include one or more of the following features. The blade-handle coupling mechanism may include an insertable coupling member and a receiving coupling member. The receiving coupling member may extend over a lip surface of the insertable coupling member to rotatably secure the insertable coupling member within a cavity defined by the receiving coupling member. The receiving coupling member may resiliently deform to securely receive the insertable coupling member into the receiving coupling member. The retractor blade may be configured for insertion into a vagina.

Another example of the present disclosure includes a medical retractor kit. An exemplary medical retractor kit may include an elongated handle member having a distal end and a proximal end, the elongated handle member configured to be held when the medical retractor is in use, the distal end of the elongated handle member having a receiving coupling member and a retractor blade configured for insertion into a body cavity. The retractor blade has a distal end and a proximal end, the proximal end of the retractor blade may have an insertable coupling member that, when coupled to the receiving coupling member, forms a blade-handle coupling mechanism that rotatably couples a distal end of the elongated handle member and to a proximal end of the retractor blade. The medical retractor kit may further include kit packaging and at least one of a lubricant and a cleaning pad.

Another exemplary medical retractor kit may include an elongated handle member having a distal end and a proximal end, the elongated handle member configured to be held when the medical retractor is in use, the distal end of the elongated handle member having an insertable coupling member, and a retractor blade configured for insertion into a body cavity. The retractor blade has a distal end and a proximal end, the proximal end of the retractor blade having a receiving coupling member that, when coupled to the insertable coupling member forms a blade-handle coupling mechanism that rotatably couples a distal end of the elongated handle member and to a proximal end of the retractor blade. This medical retractor kit may further include kit packaging and at least one of a lubricant and a cleaning pad.

Yet another aspect of the present disclosure are methods of utilizing a medical retractor. An example of such a method includes inserting a medical retractor into position within a body cavity and rotating an elongated handle member of the medical retractor relative to a retractor blade of the medical retractor at the blade-handle coupling mechanism to configure the medical retractor with a desired angle for positioning at an opening of the body cavity. The method retractor may include an elongated handle member, the elongated handle member configured to be held when the medical retractor is in use, a retractor blade configured for insertion into a body cavity, and a blade-handle coupling mechanism that rotatably couples a distal end of the elongated handle member and to a proximal end of the retractor blade. The exemplary method may further include performing an electrocauterization process after rotating the elongated handle member relative to the retractor blade. The rotating may be performed before the inserting or before the inserting in various embodiments.

These embodiments will be better understood by reference to the drawings and the detail description of the drawings that follow.

### BRIEF DESCRIPTION OF DRAWINGS

The drawings described herein serve to illustrate aspects of the present disclosure. Other aspects will be apparent to those of ordinary skill in the art based upon these drawings.

FIG. 1 is a perspective view of a medical retractor according to some embodiments of the present disclosure.

FIG. 2 is a side view of a medical retractor a medical retractor according to some embodiments of the present disclosure.

FIG. 3 is a top view of a medical retractor, in an open position, according to some embodiments of the present disclosure.

FIG. 4 is a bottom view of a medical retractor, in the open position shown in FIG. 3, according to some embodiments of the present disclosure.

FIG. 5 is an underside perspective view of a medical retractor, according to some embodiments of the present disclosure.

FIG. 6 is an exploded view of the medical retractor detailing an embodiment of a coupling mechanism thereof, according to some embodiments of the present disclosure.

FIG. 7 is a top view of a medical retractor, in an intermediate position, according to some embodiments of the present disclosure.

FIG. 8 is a top view of a medical retractor, in a closed position, according to some embodiments of the present disclosure.

FIGS. 9A and 9B are a side views of a medical retractor, in the closed position also shown in FIG. 8, according to some embodiments of the present disclosure.

FIG. 10 is a flowchart of a method of utilizing a medical retractor, according to some embodiments of the present disclosure.

These drawings will be better understood by those of ordinary skill in the art by reference to the following detailed description.

### DETAILED DESCRIPTION

The present disclosure relates to a medical retractor for providing access to a cavity of a human body, such as a vagina, for examination and/or intervention. The following detailed description of the present disclosure makes reference to the drawings, but is not limited by the drawings.

Embodiments of the present disclosure may address problems or inadequacies of existing medical retractors to provide benefits such as, but not limited to, improved access to the surgical site. The disclosed medical retractor may accomplish such benefits by increasing the potential configurations of the medical retractor so that an assisting clinician is able to reposition their supporting hand and arm to be less intrusive to the clinician performing the procedure. Additionally, embodiments of the present disclosure may increase safety during the performance of electrocauterization procedures by limiting the transfer of heat away from the specific locations in which it is intended to be applied. This may be particularly beneficial when the medical retractor is utilized to position or displace highly sensitive tissue such as the sidewall of a vagina.

Various embodiments may utilize polycarbonate or other materials that are non-conductive to prevent heating the medical retractor or exposing a patient to unwanted voltages or currents. As a result, the medical retractor may be less expensive and more easily recycled than existing retractors. The embodiments provide a medical retractor that is disposable or partially disposable to reduce risks of infection and to facilitate sterile procedures. In one example, the entire medical retractor may be disposable ore recyclable. In another example, the handle may be reusable (e.g., stainless steel coated with antibiotic materials that may be sterilized) with the retractor blade being replaceable. The medical retractor may be folded for easy transportation, storage, or packing. The position of the handle relative to the retractor blade may be ratcheted or repositioned to facilitate the clinicians and implementation of the medical procedure. The various embodiments provide numerous improvements for both clinicians and patients.

The medical retractor blade may be formed from an electrically and thermally non-conductive material such that the retractor blade includes at least a non-conductive outer surface which is both thermally non-conductive and electrically non-conductive. In one embodiment, the handle may be reusable with the retractor blades being replaced as needed. The non-conductive outer surface may be formed from the electrically and thermally non-conductive material, such as a polycarbonate material, a polymethyl methacrylate material, and a ceramic material. The elongated handle member may include a substantially straight portion at the proximal end and a curved portion at the distal end. The curved portion at the distal end of the elongated handle member may be J-curved or alternatively an L-curve. An axis of the substantially straight portion and an axis at a distal end of the curved portion may be approximately orthogonal. The blade-handle coupling mechanism may be configured to permit rotation of the retractor blade about an axis at a distal end of the curved portion of the elongated handle member. The blade-handle coupling mechanism may include detents that secure orientation of an axis of the elongated handle member with an axis of the retractor blade in multiple angles. The retractor blade may be formed of a non-conductive material and the elongated handle member may be formed from a material that is different than the non-conductive material of the retractor blade. The retractor blade may be curved along a longitudinal axis. The medical retractor may be packaged with at least one of a lubricant and a cleaning pad.

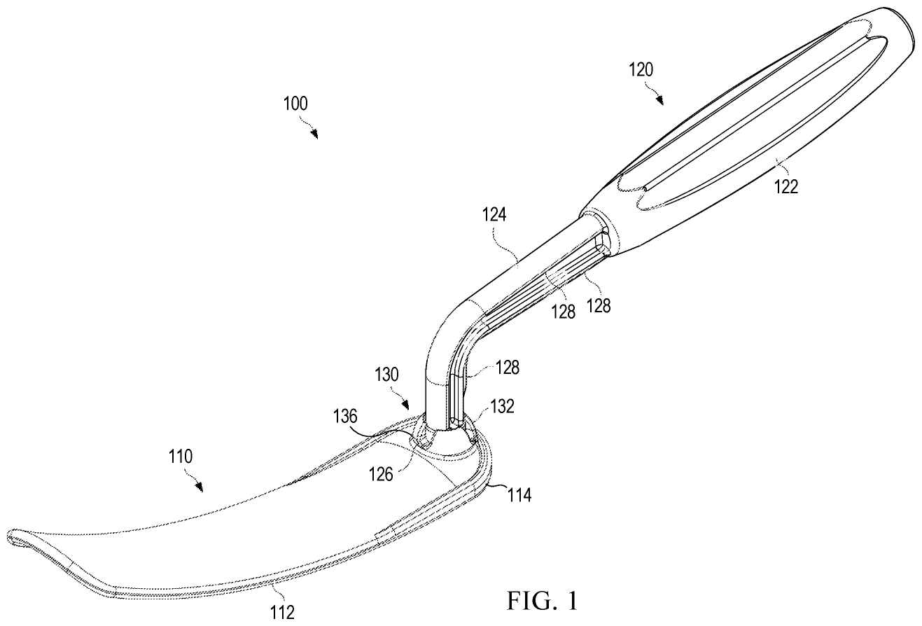

FIG. 1 is a perspective view of a medical retractor 100, according to some embodiments of the present disclosure. As shown in FIG. 1, the medical retractor 100 includes a retractor blade 110. The retractor blade 110 may be configured for insertion in a body cavity, such as a human body cavity. An example of such a cavity includes a human vagina, but other cavities, whether present in the human body naturally or formed by an injury, incision, or other surgical technique, may be advantageously accessed and maintained accessible by use of the embodiments of the medical retractor 100. Dimensions of various embodiments of the retractor blade 110 may be adapted to better suit specific cavities. The elongated handle member 120 may be sized and shaped to be held by the hand of a clinician (e.g., a surgeon, doctor, or surgical assistant) when inserting, positioning, or maintaining the medical retractor 100 in a desired position to provide access to a site of interest within the body cavity.

The medical retractor 100 includes an elongated handle member 120 rotatably coupled to the retractor blade 110. As shown in FIG. 1, the elongated handle member 120 and the retractor blade 110 are connected by a blade-handle coupling mechanism 130. As is discussed in detail through this disclosure, the blade-handle coupling mechanism 130 permits the elongated handle member 120 and the retractor blade to be reconfigured, even during in situ usage, to facilitate access to the body cavity by a clinician performing a procedure therein or therethrough.

The retractor blade 110 may be machined, milled, or injection-molded and may have a smooth or polished surface such that the retractor blade can be comfortably inserted while in contact with tissue and provide for minimal friction and abrasion while moving relative to the tissue during insertion. In some embodiments, the retractor blade 110 has a non-conductive surface formed over a substrate structure, which may or may not be conductive. In other embodiments, the retractor blade 110 is substantially formed from a non-conductive material without an underlying substrate structure of a different material. The non-conductive surface of the retractor blade 110 may be electrically and/or thermally non-conductive. Examples of suitable materials for the non-conductive retractor blade 110 include a polycarbonate material, such a polycarbonate resin thermoplastic (e.g., LEXAN® polycarbonate), a polymethyl methacrylate (PMMA) material, or a ceramic material, such as a glass or porcelain. In some embodiments, the material of the medical retractor 100 permits sterilization and reuse. However, in other embodiments, the medical retractor 100 is a disposable implement that is not intended for reuse. For example, the retractor blade 110 may be retrieved from a sterilized package (e.g., bag, plastic container, shrink wrap, etc.) before use by the clinician.

The non-conductive surface of the retractor blade 110 may limit potential complications that may occur when electrically and/or thermally conductive implements or surgical tools, such as electrocauterization tools, are used during a procedure, such as a vaginal-entry hysterectomy. In such procedures, the retractor blade 110 may be utilized to move or displace the sidewall of the vagina to provide access to the uterus. The non-conductive surface of the retractor blade 110 may prevent electrical and thermal energy from propagating through the retractor blade 110, potentially damaging, (e.g., burning, excessively heating, electrocuting, etc.) or killing the tissue in contact with the retractor blade 110 (e.g., the lateral wall or other portions of the vagina).

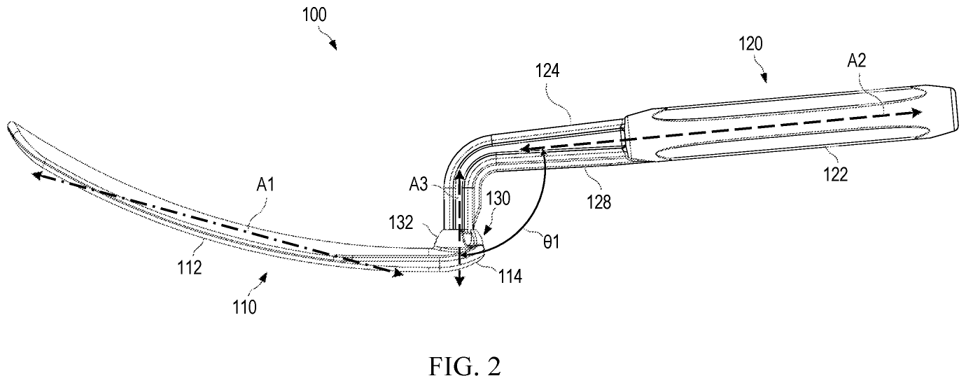

The retractor blade 110 may have a generally elongate shape. Embodiments of the retractor blade 110, such as that depicted in FIG. 1 may include a curved portion 112 having a curve or curved-shape extending along a longitudinal axis thereof. The retractor blade 110 may have a curve shape in a transverse cross-section as well, giving the retractor blade 110 a duck-bill-like shape. An example of a curved shape of the retractor blade 110 may be more clearly seen in profile in FIG. 2.

The elongated handle member 120 may be monolithic, formed from a single material by a molding, milling, or machining process, or may include multiple portions or components formed from different materials combined together into a single component. The elongated handle member 120 may include a handle portion 122 at a proximal end thereof. The handle portion 122 may include one or more ridges or fluting formed in an outer surface to be more securely held by the operator of the medical retractor 100. In some embodiments, the handle portion 122 may be rubberized or coated with an anti-slip material and/or texturized (e.g., knurled) to provide for improved grip. The handle portion 122 may also define ridges, knobs, or other shapes for the operator to securely grip and hold the handle portion 122.

The handle portion 122 may have an elongate shape as depicted in FIG. 1, in some embodiments. Other embodiments of the handle portion 122 may include a more ovoid or spherical shape, while yet other embodiments may include a flat surface to be held in the palm of the clinician's hand.

The handle portion 122 may be joined to a shaft 124 by an abutment joint or the handle portion 122 may extend over a proximal portion of the shaft 124. The handle portion 122 includes a distal end that terminates in a coupling member 126, which may be a receivable or insertable coupling member. As depicted in FIG. 1, the coupling member 126 is an insertable coupling member 126 by which the elongated handle member 120 may be rotatably secured to the retractor blade 110. The shaft 124 may have longer, generally straight portion at a proximal end thereof, and another shorter, generally straight portion at a distal end thereof, with a curved portion in between. In this way, the shaft 124 may have a curved shape, J-curved shape, or L-curved shape.

FIG. 2 is a side view of the medical retractor 100, according to some embodiments of the present disclosure. FIG. 2 more clearly depicts a curve shape of the retractor blade 110, according to some embodiments. As shown, the retractor blade 110 include a distal end and a proximal end. The distal end of the retractor blade 110 includes a curved portion 112, while the proximal end includes a substantially flat proximal portion 114. The proximal portion 114 may have a flat top surface or a flat bottom surface, or may include a flat top surface and a curved bottom surface. While the retractor blade 110 is shown as curved, the position of the retractor blade 110 relative to other components of the medical retractor 100 may be described relative to a longitudinal axis A1 of the retractor blade 110.

The top surface of the retractor blade 110 may include a blade-handle coupling mechanism 130 or may include part of the blade-handle coupling mechanism. For example, the proximal portion 114 of the retractor blade 110 may include a receiving coupling member 132 on a top surface thereof. The receiving coupling member 132 may be securely affixed to the top surface of the proximal portion 114 or may be integrally formed with the retractor blade 110.

The receiving coupling member 132 may include an opening extending fully through the retractor blade 110 or may include a cavity formed therein, extending from the top surface of the proximal portion 114 toward the bottom surface thereof. The receiving coupling member 132 is configured to rotatably receive a corresponding coupling member (insertable coupling member 126) of the elongated handle member 120, as described in further detail herein.

As can be better seen in FIG. 2, the longitudinal axis A2 of the proximal end of the shaft 124 of the elongated handle member 120 forms an angle θ1 with an axis A3 of the distal end of the shaft 124, i.e., at the insertable coupling member 126. In some embodiments, the angle θ1 may be approximately orthogonal (e.g., 90 degrees ±3, 5, 10, or 15 degrees) or in other words, the axis A2 and the axis A3 may be substantially perpendicular.

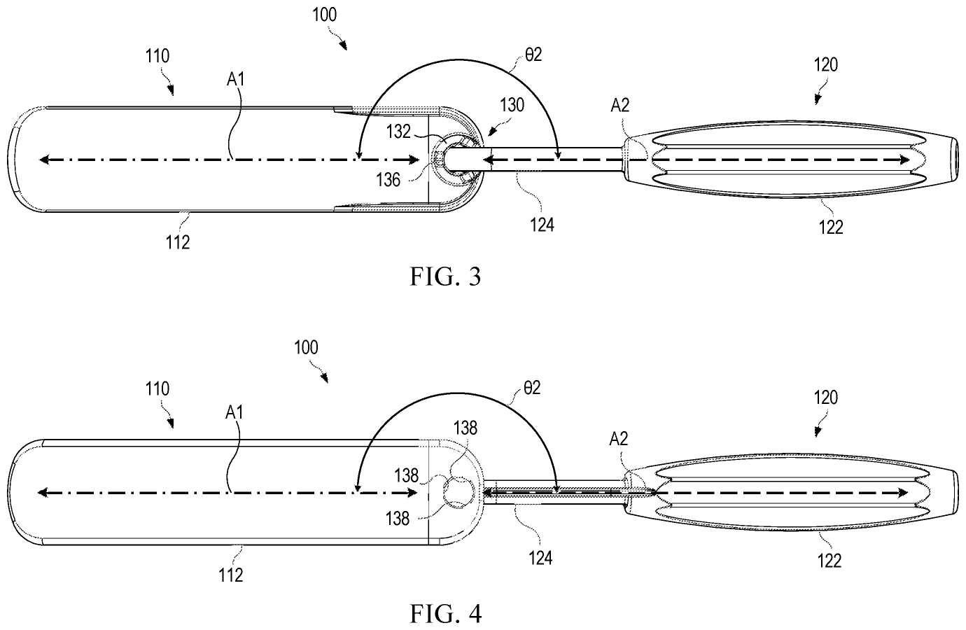

FIG. 3 is a top view of the medical retractor 100, in an open or extended position, according to some embodiments of the present disclosure. When the medical retractor 100 is in an open position or open configuration, the longitudinal axis of the retractor blade 110 (blade axis A1) is substantially aligned with the longitudinal axis of the proximal portion of the elongated handle member 120 (handle axis A2) as viewed from above.

The receiving coupling member 132 of the blade-handle coupling mechanism 130 may include one or more relief openings 136 formed in the surface of the receiving coupling member 132. In the depicted embodiments, the receiving coupling member 132 includes three such relief openings 136. The receiving coupling member 132 may be able to resiliently deform to receiving an insertable portion of the elongated handle member 120 into a central cavity of the receiving coupling member 132 and, once inserted, secure the elongate handling member 120 to retractor blade 110 while permitting rotation about an axis A3 (shown in FIG. 2). Rotation about the axis A3 permits a change in the angle θ2. As depicted in FIG. 3 and FIG. 4, the angle θ2 is approximately 180 degrees. In some embodiments, the blade-handle coupling mechanism 130 may secure the elongated handle member 120 and the retractor blade 110 together such that the elongated handle member 120 and the retractor blade 110 do not rotate freely, but rather rotate only when active pressure is applied. By applying pressure about the axis A3, a clinician operating the medical retractor 110 may be able to alter the angle θ2 to a desired position. An interference fit with interacting ridges, tabs, or extensions may also be utilized by the coupling mechanism 130.

FIG. 4 is a bottom view of the medical retractor 100, in the open position shown in FIG. 3, according to some embodiments of the present disclosure. FIG. 4 shows the distal end of the elongated handle member 120 as seen in the central cavity, which extends all the way though the retractor blade 110 in the depicted embodiments. Other embodiments may include a central cavity extending only partially through the retractor blade 110, such that the distal end of the elongated handle member 120 is not visible in the bottom view of the medical retractor 100. In yet other embodiments, the central cavity is formed by the receiving coupling member 132 without extending into the retractor blade 110.

The bottom view of the medical retractor 100 further illustrated protrusions 138. The protrusions 138 extend inwardly from a wall of the central cavity. While three such protrusions 138 are depicted, other embodiments may include more or fewer such protrusions. The protrusions 138 may extend about a millimeter inwardly in some embodiments, while extending a greater distance inwardly in other embodiments. The protrusions 138 operate to secure the distal end of the elongated handle member 120 within the central cavity of the receiving coupling member 132 by applying pressure or constraint from below while the top portion of the receiving coupling member 132 applies pressure or constraint from above. The protrusions 138 may also prevent the insertable coupling member 126 from being inserted all the way through the retractor blade 110 during assembly.

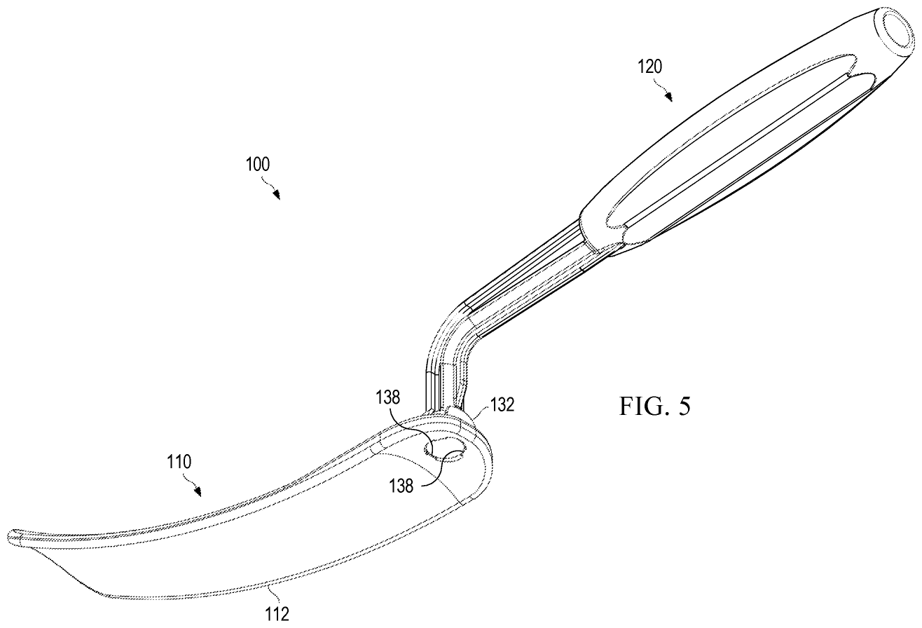

FIG. 5 is an underside perspective view of the medical retractor 100 according to some embodiments of the present disclosure. The underside perspective view provides additional views of the protrusions 138, which help to secure the instable coupling member 126 with the central cavity.

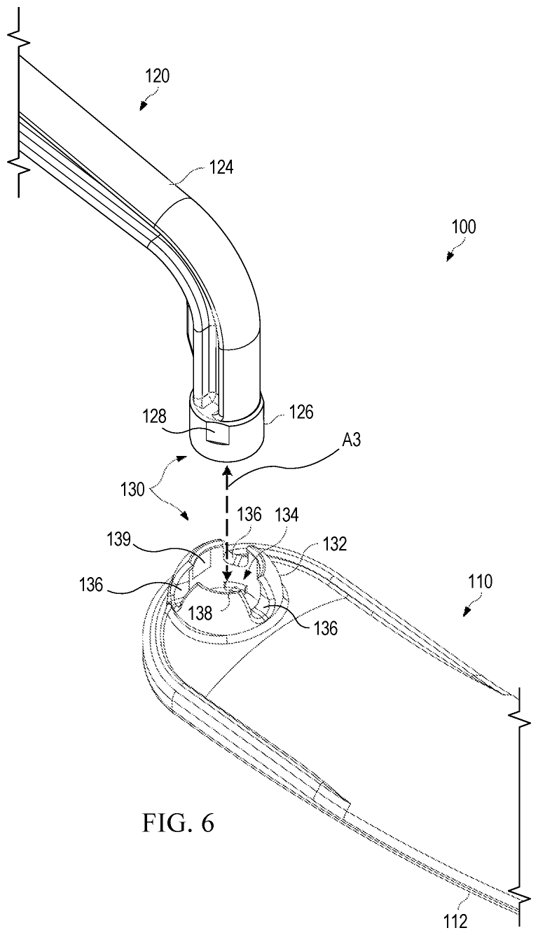

FIG. 6 is an exploded view of the medical retractor 100 detailing an embodiment of the blade-handle coupling mechanism 130 thereof, according to some embodiments of the present disclosure. As shown in FIG. 6, the retractor blade 110 and the elongated handle member 120 may be securely, rotatably coupled together by the blade-handle coupling mechanism 130. While the coupling process may take place during manufacturing of the medical retractor 100, in some embodiments the medical retractor 100 may be furnished as a kit in separate parts that may be joined prior to use by manually positioning the insertable coupling member 126 over the central cavity and applying a force to overcome resistance exerted by the top surfaces of the receiving coupling member 132. As the force is applied, the top surfaces of the receiving coupling member 132 may resiliently deform sufficiently to permit the insertable coupling member 126 to be fully inserted within the central cavity.

The receiving coupling member 126 may comprise a cylindrical shape having a larger diameter than the most proximal portion of the shorter, generally straight portion of the distal end of the shaft 124. When fully inserted, the top, inward facing surfaces of the receiving coupling member 132 extend over the top-facing surfaces or lip surfaces of the insertable coupling member 126 and apply downwardly and inwardly to stabilize the relative positions of the blade mechanism 110 and the elongated handle member 120 in a rotatable relationship. In other embodiments, the receiving coupling member 126 may be provided by a recessed portion formed around and within the distal end of the shaft 124. In some embodiments, the recessed portion may receive the top, inward facing surfaces of the receiving coupling member 132.

Other embodiments of the medical retractor 100 may provide for other mechanisms and structures for securing the retractor blade 110 and the elongated handle member 120 together while permitting them to rotate relative to each other, adjusting the angle θ2. For example, the insertable coupling member 126 may include a threaded connection on an outer surface thereof that engaged with a corresponding threaded connection on an inner surface of the central cavity.

By adjusting the angle θ2, the medical retractor 100 may be reconfigured by the clinician, while it is in situ within a body cavity, so that the elongated handle member 120 is in a position that better accommodates the use of other instruments near or within the body cavity or provides better access or visibility to the body cavity. When the medical retractor 100 is used in the performance of a hysterectomy, for example, the angle θ2 may be adjusted to permit the assisting clinician holding the medical retractor 100 to position himself or herself out of the way of the clinician performing the surgery. This adjustment can be made while the retractor blade 110 remains generally stationary within the body cavity.

The outer surface of the insertable coupling member 126 (or the adjacent area of the distal end of the shaft 124) may include one or more detents or, more broadly, detent structures 128 (one is depicted in FIG. 6) that cooperate with corresponding detent structures 139 on or in the receiving coupling member 132 to provide for the angle θ2 to be secured in a one or more predetermined angles. The detent structures 128 and 139 may secure the orientation of the elongated handle member 120 and the retractor blade 110 in the angle θ2. In some embodiments, the one or more detent structures 128 protrude outwardly from the exterior surface of the insertable coupling member 126 and may interact with the openings 136 formed in the receiving coupling member 132.

For example, various embodiments of the detent structures 128 (on an exterior surface of the insertable coupling member 126) and detent structures 139 (on an interior surface of the central cavity and/or on interior surface of the receiving coupling member 132) may be positioned such the medical retractor 100 can be securely utilized with the angle θ2 is a predetermined angle or angles including one or of the following exemplary angles: ±60 degrees, ±90 degrees, ±120 degrees, 135 degrees, ±150 degrees, 180 degrees, within ordinary manufacturing tolerances. Other predetermined configurations of detent structures 128 and 139 may provide for other angles. For example, the configuration of the medical retractor 100 in FIGS. 3 and 4, in which the angle θ2 is approximately 180 degrees, may be securely maintained during use because of the detent structures 128 and 139. In some embodiments, the medical retractor 100 may be maintained in the open or extended configuration (angle θ2=180) until an intentional force is applied by the clinician to overcome the resistance provided by the detent structures 128 and 139. As a result, the position of the retractor blade 110 relative to the elongated handle member 120 may be adjusted or ratched as needed to effectively adjust to the needs of the clinician and procedure being implemented.

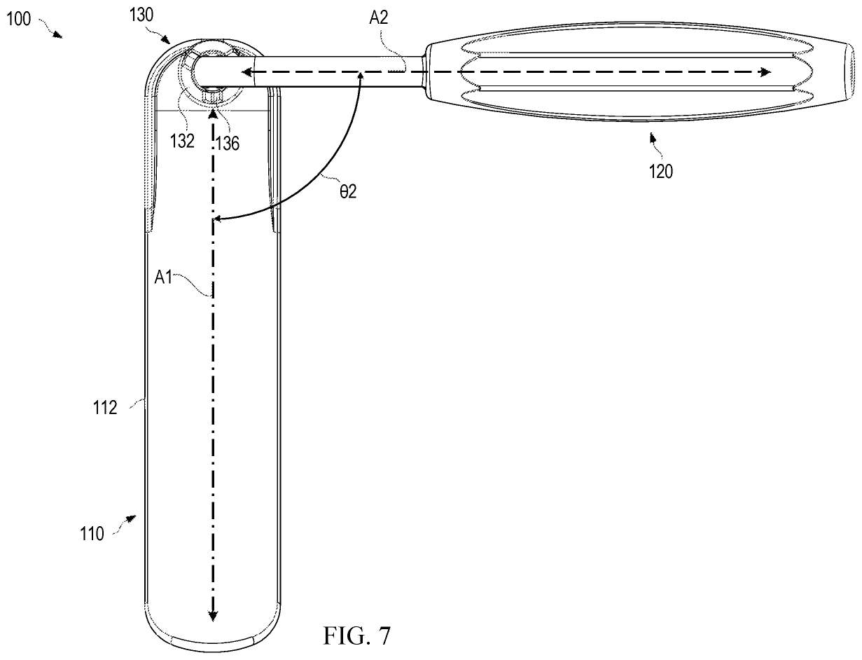

FIG. 7 is a top view of the medical retractor 100, in an intermediate position, according to some embodiments of the present disclosure. FIG. 7 shows the medical retractor 100 in a 90-degree configuration, i.e., a configuration in which angle θ2 is approximately 90 degrees. During use of the medical retractor 100, the operator of the medical retractor 100 may insert the retractor blade 110 into a body cavity, such as a vagina, to apply a force to retract or displace the sidewall of the vagina to provide access therein. Thereafter, in order to better access the body cavity, the operator of the medical retractor 100 may secure the retractor blade 110 in position with one hand and with the other hand apply sufficient force to rotate the elongated handle member 120 about the axis A3.

The elongated handle member 120 may be rotated about the axis A3 approximately 45, 60, 90, or more degrees, in some embodiments. While rotating, the elongated handle member 120 may engage and disengage one or more detent structures. After the medical retractor 100 has been configured with a desired angle θ2, a medical procedure may be performed or a medical implement may be utilized to visualize, manipulate, or treat tissue located in the body cavity or made accessible through the body cavity. During a hysterectomy, for example, an operator of the medical retractor 100 may insert the retractor blade 110 to retract the sidewall of the vagina, adjust the angle θ2 to provide improved accessibility, and then another clinician may perform an electrocauterization process during the procedure. Because the medical retractor 100 may be adjusted after insertion, the electrocauterization process may be more efficaciously performed. Additionally, because of the non-conductive material of at least the outer surface of the retractor blade 110, the risk of transferring heat energy or electrical energy to the tissues in contact with the retractor blade 110 (e.g., the delicate sidewall tissues of the vagina) may be minimized or eliminated.

In some instances, the operator of the medical retractor 100 may reconfigure it after the procedure is completed, such that the medical retractor 100 is returned to the open or extended configuration for removal from the body cavity. In some embodiments, the medical retractor 100 may be removed from the body cavity without being returned by the operator to the open configuration.

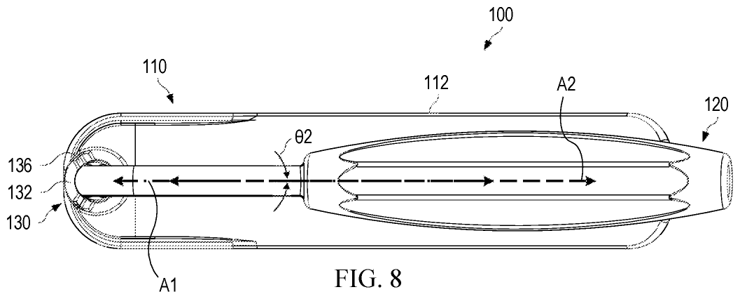

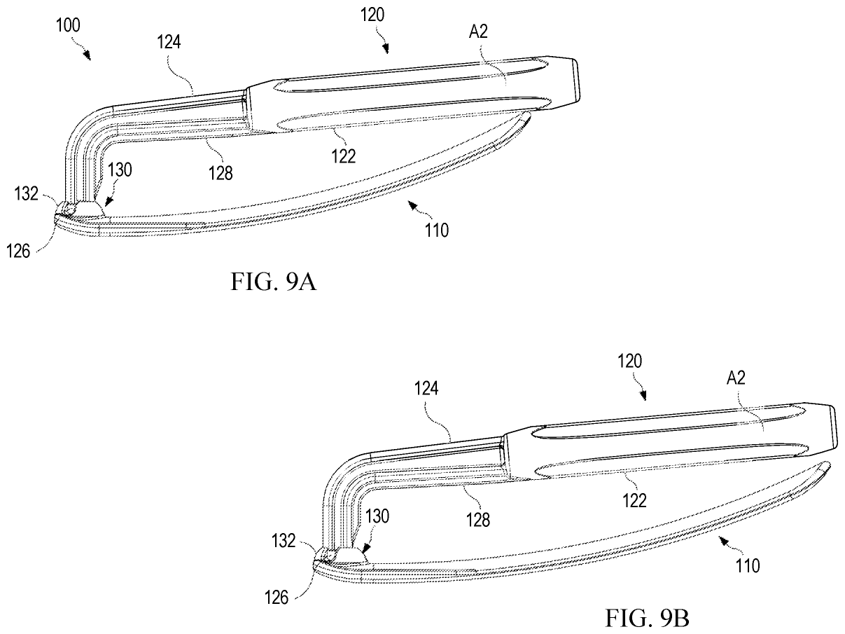

FIG. 8 is a top view of the medical retractor 100 and FIGS. 9A and 9B are side views of the medical retractor 100, in a closed position, according to some embodiments of the present disclosure. The medical retractor 100 may be manipulated such that the angle θ2 is approximately 0 degrees. In this configuration, the medical retractor 100 may be packaged for shipping and/or stored while occupying less volume or space. Thus, the medical retractor 100 may be shipped more affordably or may be stored in larger quantities in the same space. This may be particularly advantageous when the elongated handle member 120 and the retractor blade 110 are transported in a joined state or when the elongated handle member 120 and the retractor blade 110 cannot be easily joined together just prior to use.

As shown in FIGS. 9A and 9B, the curvature of the retractor blade 110, and the dimensions of the elongated handle member 120 may be such that when the medical retractor is in the closed configuration, the distal end of the retractor blade 110 and the proximal end of the elongated handle member 120 may not be in direct contact unless acted upon by an external force (FIG. 9A). However, when a force is applied (such as by tight packaging or vacuum packaging) the distal end of the retractor blade 110 and the proximal end of the elongated handle member 120 may be brought into contact. In other embodiment, the curvature of the retractor blade 110 and the dimensions of the elongated handle member 120 may be such that when the medical retractor 100 is in the closed position the retractor blade 110 may be resiliently deformed slightly by the proximal end of the elongated handle member 120 (FIG. 9B).

Embodiments of the present disclosure include a packaged medical retractor kit, that includes at least the elongated handle member 120 and the retractor blade 110. In such a medical retractor kit, the elongated handle member 120 and the retractor blade 110 may be provided in a disconnected state. The kit may include the elongated handle member 120 and the retractor blade 110 in a sterile packaging that can be opened in the environment in which a medical procedure is to be performed. In some embodiments, after the kit has been opened and the elongated handle member 120 and the retractor blade 110 removed, an insertable coupling member of the elongated handle member 120 may be inserted into the receiving coupling member of the retractor blade 110. In other embodiments, an insertable coupling member of the retractor blade 110 may be inserted into a receiving coupling member of the elongated handle member 120.

Embodiments of the packaged medical retractor kit, the package may further include items that may be used in connection with the medical retractor 100. For example, the packaged medical retractor kit may include a lubricant that may be used to coat the retractor blade 110, alcohol preparation or cleaning pads for cleaning and preparing for a procedure, and other items apparent to one of ordinary skill in the art.

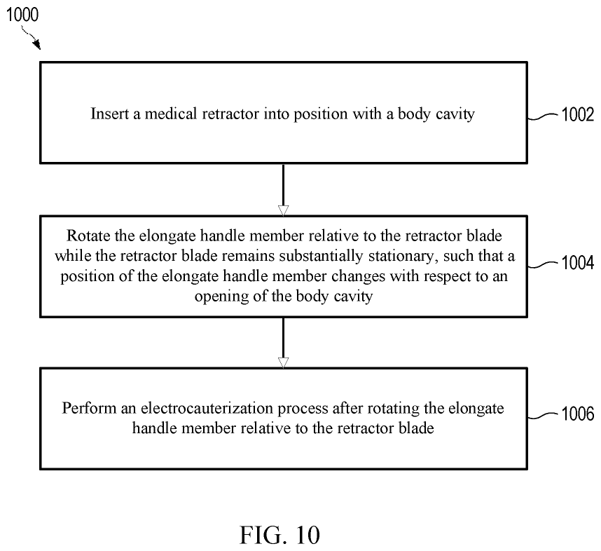

FIG. 10 is a flowchart of a method 1000 of utilizing the medical retractor 100. As depicted in FIG. 10, the method 1000 may include an operation 1002 of inserting a medical retractor into position with a body cavity. This may be done by inserting the retractor blade 110 into the body cavity, such as a vagina. After the retractor blade 110 has been inserted to a satisfactory position, the method includes an operation 1004 of rotating the elongated handle member relative to the retractor blade 110 while the retractor blade 110 remains substantially stationary or is further positioned within the body cavity. The medical retractor may be used to displace or retract the sidewall of the body cavity, such as a vagina, to facilitate a medical procedure.

In operation 1004, a position of the elongated handle member 120 is changed with respect to an opening of the body cavity. For example, the elongated handle member 120 of the medical retractor 100 may be moved away from the body cavity to provide better access to the body cavity for a diagnostic or surgical procedure that may be performed as operation 1006.

In some embodiments of the method 1000, the operation 1004 may be performed prior to the operation 1002, e.g., the medical retractor 100 may be manipulated to have a desired angle θ2 before insertion into the body cavity.

As an example of operation 1006, an exemplary surgical procedure may include a hysterectomy that includes an electrocauterization process after rotating the elongated handle member relative to the retractor blade (operation 1004) and inserting the medical retractor into the body cavity (operation 1002). The results of procedures may be improved my providing the improved access to the site and/or by the thermally and/or electrically non-conductive properties of the retractor blade 110, which may reduce or eliminate heating of the blade 110, which in turn may reduce or eliminate irritation or damage to the tissues being positioned using the medical retractor 100. In some embodiments of the method 1000, an additional embodiment of the medical retractor 100 may be utilized during any procedure in a manner similar to that describe in operations 1002 and 1004.

The scope of this disclosure is not limited to the specific embodiments disclosed. Additional modifications may be apparently to one of ordinary skill in the art in view of the description, figures, and claims. Such modifications are also within the scope of this disclosure.

What is claimed is:

1. A medical retractor comprising:
an elongated handle member having a distal end and a proximal end, the elongated handle member configured to be held when the medical retractor is in use;
a retractor blade configured for insertion into a body cavity, the retractor blade having a distal end and a proximal end; and
a blade-handle coupling mechanism that rotatably couples a distal end of the elongated handle member and to a proximal end of the retractor blade to permit a change in an angle between the elongated handle member and the retractor blade while the retractor blade is positioned within the body cavity.

2. The medical retractor of claim 1, wherein the retractor blade is formed from an electrically and thermally non-conductive material such that the retractor blade comprises at least a non-conductive outer surface which is both thermally non-conductive and electrically non-conductive.

3. The medical retractor of claim 2, wherein the non-conductive outer surface is formed from the electrically and thermally non-conductive material selected from a set consisting of a polycarbonate material, a polymethyl methacrylate material, and a ceramic material.

4. The medical retractor of claim 1, wherein the elongated handle member comprises a substantially straight portion at the proximal end and a curved portion at the distal end.

5. The medical retractor of claim 4, wherein the curved portion at the distal end comprises a J-curve.

6. The medical retractor of claim 4, wherein an axis of the substantially straight portion and an axis at a distal end of the curved portion are approximately orthogonal.

7. The medical retractor of claim 4, wherein the blade-handle coupling mechanism is configured to permit rotation of the retractor blade about an axis at a distal end of the curved portion of the elongated handle member.

8. The medical retractor of claim 1, wherein the blade-handle coupling mechanism comprises a plurality of detents that secure orientation of an axis of the elongated handle member with an axis of the retractor blade in a plurality of angles.

9. The medical retractor of claim 1, wherein the retractor blade is formed of a non-conductive material and the elongated handle member is formed from a material that is different than the non-conductive material of the retractor blade.

10. The medical retractor of claim 1, wherein the retractor blade is curved in a transverse cross-section.

11. The medical retractor of claim 1, wherein the retractor blade is a curved along a longitudinal axis.

12. A medical retractor of claim 1, wherein the medical retractor is packaged with at least one of a lubricant and a cleaning pad.

13. A method of utilizing the medical retractor of claim 1, the method comprising:
inserting the medical retractor into position relative to the body cavity; and
rotating the elongated handle member relative to the retractor blade at the blade-handle coupling mechanism to configure the medical retractor with desired angle for positioning at an opening of the body cavity.

14. The method of claim 13 further comprising performing an electrocauterization process after rotating the elongated handle member relative to the retractor blade.

15. A medical retractor comprising:
an elongated handle member having a distal end and a proximal end, the elongated handle member configured to be held when the medical retractor is in use;
a retractor blade configured for insertion into a body cavity, the retractor blade having a distal end and a proximal end; and
a blade-handle coupling mechanism that rotatably couples a distal end of the elongated handle member and to a proximal end of the retractor blade to permit a change in an angle between the elongated handle member and the retractor blade while the retractor blade is positioned within to the body cavity;
wherein the retractor blade is formed from an electrically and thermally non-conductive material such that the retractor blade comprises at least a non-conductive outer surface which is both thermally non-conductive and electrically non-conductive;
wherein the elongated handle member comprises a substantially straight portion at the proximal end and a curved portion at the distal end.

16. The medical retractor of claim 15, wherein the non-conductive outer surface is formed from the electrically and thermally non-conductive material selected from a set consisting of a polycarbonate material, a polymethyl methacrylate material, and a ceramic material.

17. The medical retractor of claim 15 wherein the curved portion at the distal end comprises a J-curve.

18. The medical retractor of claim 15, wherein the blade-handle coupling mechanism comprises a plurality of detents that secure orientation of an axis of the elongated handle member with an axis of the retractor blade in a plurality of angles.

19. The medical retractor of claim 15, wherein the retractor blade is curved in a transverse cross-section.

20. The medical retractor of claim 15, wherein the retractor blade is a curved along a longitudinal axis.
